(19) United States
(12) Patent Application Publication
(10) Pub. No.: US 2006/0201512 A1
(43) Pub. Date: Sep. 14, 2006

(54) Nasal cannula

(76) Inventors: Michael Ernest Garrett, Woking Surrey (GB); Geoffrey Morgan Lloyd, Farnborough Hants (GB); Thomas Rathbone, Farnham Surrey (GB)

Correspondence Address: THE BOC GROUP, INC., 575 MOUNTAIN AVENUE, MURRAY HILL, NJ, 07974-2064, US

(21) Appl. No.: 10/543,025

(22) PCT Filed: Jan. 29, 2004
(86) PCT No.: PCT/GB04/00352

§ 371 Date: Mar. 30, 2006

(30) Foreign Application Priority Data
Jan. 30, 2003 (GB) 0302196.1

(51) Int. Cl. A62B 7/10; A61M 15/08

(52) U.S. Cl. 128/206.11; 128/207.18

(57) ABSTRACT

A nasal cannula 1 for inclusion in medical apparatus that delivers a breathable gas mixture of helium and oxygen to a patient comprises a length 12 of high pressure narrow bore tubing having a proximal end region for connection to a high pressure source, a cylinder 2, of the breathable gas mixture at a junction 8. The length 12 of tubing has a distal end region connected to at least one nasal administration device 10. The distal end region of the length 12 of tubing has an orifice for the expansion of the breathable gas mixture.

The present invention relates to medical apparatus and cannula for delivering a breathable gas mixture to a patient.

It is known to administer oxygen to a patient so as to facilitate their breathing.

It is also known to deliver helium/oxygen gas mixtures to a patient with breathing difficulties to reduce airway resistance and improve lung ventilation. Known methods of delivery utilise one or more cylinders of gas. Each cylinder has associated with it a two stage pressure regulator to reduce the pressure of the gas from a high storage pressure (typically in the order of 200 bar) to near atmospheric pressure. The gas flows through relatively wide diameter tubing to an administration device, for example a nasal cannula. The gas is administered to the patient at room temperature. An example of a known nasal cannula for operation of approximately atmospheric pressure is disclosed in GB-A-1 081 807.

It is an aim of the present invention to provide an improved apparatus for delivering a breathable gas mixture including helium to a patient which does not require plural stage gas regulation.

According to the present invention there is provided a nasal cannula for delivering a breathable gas mixture comprising helium and oxygen to a patient, the nasal cannula comprising a length of high pressure narrow bore tubing having a proximal end region for connection to a high pressure source of the breathable gas mixture and a distal end region connected to at least one nasal administration device, wherein the nasal administration device or the distal end region of the tubing has at least one orifice for the expansion of the breathable gas mixture.

The invention also provides apparatus for administering a breathable gas mixture comprising helium and oxygen including means for supplying the breathable gas mixture at a high pressure and a nasal cannula according to the invention.

The nasal cannula and apparatus according to the invention offer the following advantages. First, the requirement for plural stage gas regulation to reduce the supply pressure to near atmospheric pressure is eliminated. Typically, a single stage regulator is used to supply the breathable gas at a pressure in the range of 100 to 300 bar. At such high supply pressures, the cannula tubing need only have a narrow bore. Therefore, unsightly wide bore tubing is avoided.

The breathable gas mixture is preferably a helium-oxygen mixture containing from, say, 20 to 30% by volume of oxygen (and hence 70 to 80% by volume of helium). Every gas has a Joule-Thomson inversion temperature. Above the inversion temperature throttling of the gas through an expansion orifice leads to its heating. Below the inversion temperature such throttling of the gas leads to its cooling. At ambient temperature oxygen is cooled on being expanded, but helium is warmed. The pressure at which the cannula receives the breathable gas and the composition of the mixture may thus be selected to give a desired degree of warming to the gas mixture on expansion through the said orifice. Indeed, an inhaled gas at close to the ideal tracheal temperature of the human body can be formed. Such a temperature facilitates a patient's breathing.

The breathable mixture of helium and oxygen may be stored at the desired high pressure in a gas cylinder.

The high-pressure tubing may be coiled.

The high-pressure tubing may be of a ductile metal or alloy, for example a cupro-nickel alloy.

The high pressure tubing may be surrounded by a protective sheath. The protective sheath may be of plastic material.

The nasal administration device may be a device defining a nasal prong or pair of nasal prongs.

An embodiment of the invention will now be described by way of example, reference being made to the Figures of the accompanying diagrammatic drawings in which:

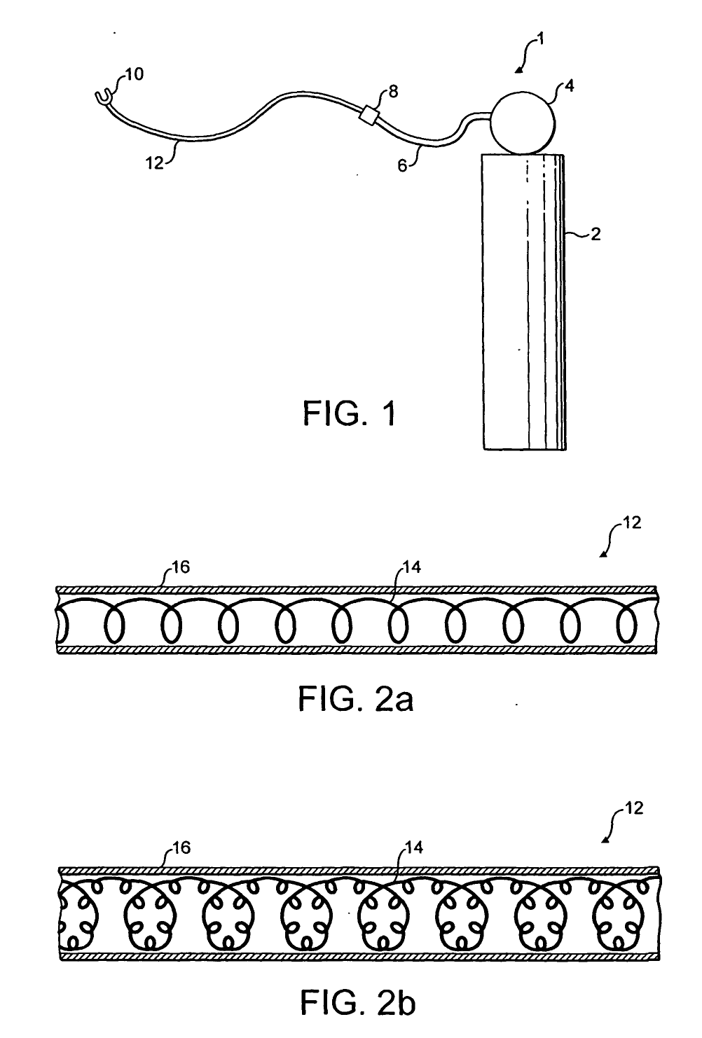

FIG. 1 is a schematic view of an apparatus for delivering a breathable gas mixture under pressure to a patient according to the present invention;

FIGS. 2a and 2b are schematic details of a coiled high pressure line forming part of the apparatus of FIG. 1; and

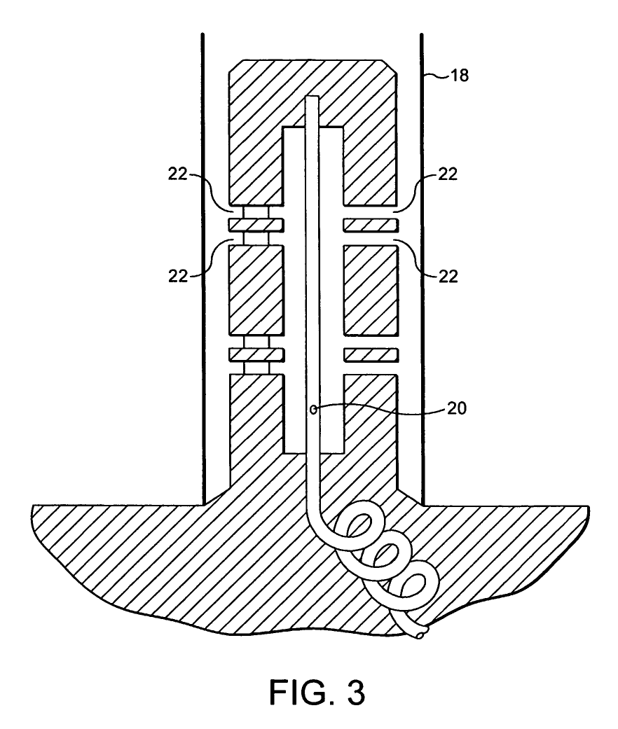

FIG. 3 is a schematic detail of an end of a coiled high pressure line terminating at a tubular nasal prong forming part of the nasal cannula forming part of the apparatus of FIG. 1.

As shown in FIG. 1, an apparatus 1 for delivering a breathable gas mixture including helium under pressure to a patient comprises a source of the gas mixture in the form of a cylinder 2 to which is mounted a single stage gas regulator 4 in a manner known per se. Preferably, the mixture contains 28% by volume helium, the remainder being oxygen.

A high pressure flexible braided hose 6 extends from the regulator 4 to a junction 8. A nasal cannula 7 is connected at its proximal end to the junction 8. The cannula 7 comprises a coiled high pressure tubing 12 and a nasal gas administration device 10.

Referring also to FIGS. 2a, 2b the coiled high pressure tubing 12 comprises a coiled high pressure cupro-nickel tube 14 having an internal diameter of between 10 and 15 thousandths of an inch enclosed within a tubular flexible protector sheath 16.

The coiled high pressure tubing 12 terminates at its distal end region in a tubular nasal prong or prongs 18 (see FIG. 3) forming part of the nasal cannula 10 where it is formed with an expansion orifice 20. The tubular nasal prong 18 is formed with a plurality of perforations 22 which allow the flow of the gas mixture from the cannula 10 in to the nasal passages of a patient.

In use, the gas mixture under high pressure leaves the cylinder 2 and passes through the regulator 4. The pressure at the outlet of the regulator 4 is preferably in the range 100 bar to 300 bar. The gas mixture passes via the high pressure flexible braided hose 6 to the cannula 7.

The gas mixture leaves the coiled high pressure tubing 12 of the cannula 7 through the expansion orifice 20 and is thereby expanded from the pressure in the range of 100 bar to 300 bar to a pressure that is approximately atmospheric pressure. The atmospheric pressure gas passes through the perforations 22 for passage in to the nasal cavities of a patient.

A particular advantage of the embodiment described above is that if a mixture of oxygen and helium is expanded the combined Joule Thomson effect of expanding both helium and oxygen is one of heating. The gas is thus raised in temperature by a few degrees Celsius. In the preferred mixture of 28% helium in oxygen the temperature of gas can be raised approximately 14° C. above ambient yielding an inhaled gas which is close to the ideal tracheal temperature of the body. Thus the patient is provided with a slightly warmed gas with enhanced oxygen and easy breathability and the pressure of the gas mixture precludes any possibility of blockage by bodily fluids.

As modelled with HYSYS v 3.0.1 software using Peng Robinson with Lee-Kessler mixing rules a 28% by volume oxygen, 72% by volume helium mixture shows the following rise in temperature following an expansion to atmospheric pressure when the mixture is received at 20° C. for expansion.

| | | | |
|---|---|---|---|
| Upstream pressure (Bar) | 300 | 200 | 100 |
| Joule Thomson temperature rise (K) | 13.6 | 8.3 | 3.6 |

1. A nasal cannula for delivering a breathable gas mixture comprising helium and oxygen to a patient, the nasal cannula comprising a length of high pressure narrow bore tubing having a proximal end region for connection to a high pressure source of the breathable gas mixture at a pressure in the range of 100 bar to 300 bar and a distal end region connected to at least one nasal administration device, wherein the nasal administration device or the distal end region of the tubing has at least one orifice for the expansion of the breathable gas mixture.

2. The nasal cannula according to claim 1, wherein the high pressure narrow bore tubing is coiled.

3. The nasal cannula according to claim 1, wherein the high pressure narrow bore tubing is of a ductile metal or alloy.

4. The nasal cannula according to claim 3, wherein the alloy is a cupro-nickel alloy.

5. A The nasal cannula according to claim 1, wherein the high pressure narrow bore tubing is surrounded by a protective sheath.

6. A The nasal cannula according to claim 1, in which the nasal administration device defines a nasal prong or a pair of nasal prongs.

7. An apparatus for administering a breathable gas mixture comprising helium and oxygen including means for supplying the breathable gas mixture at a high pressure and a nasal cannula comprising a length of high pressure narrow bore tubing having a proximal end region for connection to a high pressure source of the breathable gas mixture at a pressure in the range of 100 bar to 300 bar and a distal end region connected to at least one nasal administration device, wherein the nasal administration device or the distal end region of the tubing has at least one orifice for the expansion of the breathable gas mixture.

8. The apparatus according to claim 7, wherein the said means includes a gas cylinder in which the breathable gas mixture is stored under pressure.

9. The apparatus according to claim 8, wherein the stored breathable gas mixture contains from 70 to 80% by volume of helium and from 20 to 30% by volume of oxygen.

10. The apparatus according to claim 9, wherein the stored breathable gas mixture is stored in the cylinder at a pressure in the range of 100 bar to 300 bar.

11. The apparatus according to claim 8, wherein the stored breathable gas mixture contains 72% by volume of helium, balance oxygen.
